(19) United States
(12) Patent Application Publication
(10) Pub. No.: US 2026/0151587 A1
(43) Pub. Date: Jun. 4, 2026

(54) Continuous Positive Airway Aromatherapy Device

(71) Applicant: Jacob Montgomery, Waverly, OH (US)

(72) Inventor: Jacob Montgomery, Waverly, OH (US)

(21) Appl. No.: 19/010,303

(22) Filed: Jan. 6, 2025

Related U.S. Application Data
(60) Provisional application No. 63/727,307, filed on Dec. 3, 2024.

(51) Int. Cl. A61M 21/02; A61M 16/00; A61M 21/00

(52) U.S. Cl. CPC A61M 21/02; A61M 16/0003; A61M 2021/0016; A61M 2202/0007; A61M 2202/02; A61M 2205/0216; A61M 2205/025; A61M 2205/05; A61M 2205/12; A61M 2205/583

(57) ABSTRACT

A continuous positive airway aromatherapy device is provided that is designed to integrate with CPAP machines, providing users with therapeutic aromatherapy during sleep therapy. The device comprises a medical-grade body with a first end for secure attachment to the CPAP machine's air intake port and a second end connecting to CPAP tubing, both utilizing airtight fasteners. The interior of the body is comprised of a receiving area for replaceable fragrance cartridges, which hold medical-grade essential oils or therapeutic blends. A hinged or snap-fit lid ensures the cartridge is securely enclosed while maintaining an airtight seal with the aid of elastic gaskets. During operation, the device channels CPAP airflow over the fragrance cartridge, diffusing scent molecules into the user's airflow, enhancing the therapy experience.

### CROSS-REFERENCE TO RELATED APPLICATION

The present application claims priority to, and the benefit of, U.S. Provisional Application No. 63/727,307, which was filed on Dec. 3, 2024, and is incorporated herein by reference in its entirety.

### FIELD OF THE INVENTION

The present invention relates generally to the field of continuous positive airway aromatherapy devices. More specifically, the present invention relates to an aromatherapy device for CPAP machines, featuring a medical-grade body with airtight connections to integrate with a CPAP machine and tubing. The device is also comprised of a receiving area for replaceable fragrance cartridges, which diffuse therapeutic scents into the airflow to enhance the user's CPAP experience. Accordingly, the present disclosure makes specific reference thereto. Nonetheless, it is to be appreciated that aspects of the present invention are also equally applicable to other like applications, devices, and methods of manufacture.

### BACKGROUND

Continuous Positive Airway Pressure (CPAP) machines are essential medical devices used to treat individuals suffering from sleep apnea and other respiratory conditions. These machines deliver a steady stream of air to keep the airways open, enabling users to maintain uninterrupted breathing during sleep. For individuals relying on CPAP therapy, consistent and efficient performance of the machine is critical to their health and well-being. However, the benefits of CPAP therapy can be significantly reduced when the user is experiencing nasal congestion or a cold. These conditions cause obstructions in the nasal passages, making it difficult for the user to receive the full airflow provided by the machine. The discomfort caused by congestion can disrupt sleep, deter users from consistent CPAP usage, and compromise the effectiveness of the therapy. This situation not only affects the quality of sleep but may also lead to secondary health issues, such as increased fatigue, cardiovascular strain, and exacerbation of respiratory problems. Despite the widespread adoption of CPAP machines, there remains a gap in addressing these challenges in a way that is both effective and user-friendly.

Therefore, there exists a long-felt need in the art for a continuous positive airway aromatherapy device that enhances the respiratory benefits of a CPAP machine by reducing nasal congestion. There also exists a long-felt need in the art for a continuous positive airway aromatherapy device that introduces therapeutic fragrances to improve user comfort during CPAP therapy. Moreover, there exists a long-felt need in the art for a continuous positive airway aromatherapy device that seamlessly integrates with existing CPAP systems without interfering with their operational efficiency.

The subject matter disclosed and claimed herein, in one embodiment thereof, comprises a continuous positive airway aromatherapy device designed to integrate with CPAP machines, providing users with therapeutic aromatherapy during sleep therapy. The device comprises a medical-grade body with a first end for secure attachment to the CPAP machine's air intake port and a second end connecting to CPAP tubing, both utilizing airtight fasteners. The interior of the body is comprised of a receiving area for replaceable fragrance cartridges, which hold medical-grade essential oils or therapeutic blends. A hinged or snap-fit lid ensures the cartridge is securely enclosed while maintaining an airtight seal with the aid of elastic gaskets. During operation, the device channels CPAP airflow over the fragrance cartridge, diffusing scent molecules into the user's airflow, enhancing the therapy experience.

In this manner, the continuous positive airway aromatherapy device of the present invention accomplishes all the forgoing objectives and provides a device that attaches directly to a CPAP housing and features an input port, an output port, and a slot configured to hold interchangeable fragrance cartridges, such as menthol, peppermint, or lavender, which can alleviate congestion and enhance relaxation. The snap lid ensures an airtight seal, maintaining the performance of the CPAP machine while allowing for easy cartridge replacement. By integrating therapeutic aromas into the airflow, the device provides users with a more comfortable and effective respiratory therapy experience, particularly during times of nasal congestion. This innovation not only improves user satisfaction but also supports consistent CPAP usage by addressing the challenges associated with nasal obstruction, thus enhancing overall health outcomes.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a continuous positive airway aromatherapy device. The device is designed to integrate with CPAP machines, providing therapeutic aromatherapy to enhance the user's sleep therapy experience. The body of the device serves as the primary housing, comprising and protecting internal components, and is constructed from medical-grade materials such as ABS plastic, polycarbonate, or biocompatible polymers.

The first end of the body connects securely to the CPAP machine's air intake port using an input fastener that may include threaded connectors, clamps, or snap-fit mechanisms. Alternative fastener designs, such as bayonet mounts or quick-connect couplings, ensure an airtight and reliable connection. The second end interfaces with the CPAP tubing through an output fastener, utilizing compression fittings, threaded connectors, or friction locks to maintain a secure and leak-free attachment.

An interior space within the body directs airflow while maintaining the pressure and flow consistency necessary for CPAP therapy. The interior contains a receiving area designed to hold a replaceable fragrance cartridge securely. This cartridge contains hypoallergenic, medical-grade fragrances, such as essential oils, formulated for respiratory relief or relaxation. Fragrance diffusion is controlled through openings in the cartridge, such as porous membranes or vent systems.

The receiving area is covered by a lid that provides an airtight seal using elastic, hypoallergenic materials. The lid is secured via a locking mechanism, which may be a snap-fit latch, magnetic closure, or threaded ring. In some embodiments, the lid is transparent or translucent, allowing users to monitor the cartridge status.

During operation, the device connects to the CPAP machine and tubing, with air flowing through the interior space, passing over the fragrance cartridge. Scent molecules are diffused into the airflow and delivered to the user, enhancing the therapeutic experience. The fragrance cartridge can be easily replaced when depleted, minimizing therapy disruption.

The invention also includes a method of use, involving the steps of connecting the device to a CPAP machine and tubing, inserting the fragrance cartridge, securing the lid, and operating the CPAP machine to diffuse the fragrance. Cartridge replacement is designed to be quick and intuitive, ensuring continued functionality and ease of use.

Accordingly, the continuous positive airway aromatherapy device of the present invention is particularly advantageous as it provides a device that attaches directly to a CPAP housing and features an input port, an output port, and a slot configured to hold interchangeable fragrance cartridges, such as menthol, peppermint, or lavender, which can alleviate congestion and enhance relaxation. The snap lid ensures an airtight seal, maintaining the performance of the CPAP machine while allowing for easy cartridge replacement. By integrating therapeutic aromas into the airflow, the device provides users with a more comfortable and effective respiratory therapy experience, particularly during times of nasal congestion. This innovation not only improves user satisfaction but also supports consistent CPAP usage by addressing the challenges associated with nasal obstruction, thus enhancing overall health outcomes. In this manner, the continuous positive airway aromatherapy device overcomes the limitations of existing CPAP machines known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a continuous positive airway aromatherapy device of the present invention attached to a CPAP machine in accordance with the disclosed architecture;

FIG. 2 illustrates an exploded perspective view of one potential embodiment of a continuous positive airway aromatherapy device of the present invention in accordance with the disclosed architecture;

FIG. 3 illustrates a side exploded perspective view of one potential embodiment of a continuous positive airway aromatherapy device of the present invention in accordance with the disclosed architecture; and

FIG. 4 illustrates a flowchart of a method of using one potential embodiment of a continuous positive airway aromatherapy device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a continuous positive airway aromatherapy device that enhances the respiratory benefits of a CPAP machine by reducing nasal congestion. There also exists a long-felt need in the art for a continuous positive airway aromatherapy device that introduces therapeutic fragrances to improve user comfort during CPAP therapy. Moreover, there exists a long-felt need in the art for a continuous positive airway aromatherapy device that seamlessly integrates with existing CPAP systems without interfering with their operational efficiency.

The present invention, in one exemplary embodiment, is comprised of a continuous positive airway aromatherapy device. The device integrates with CPAP machines to provide therapeutic aromatherapy, enhancing the user's sleep therapy experience. The body of the device functions as the primary housing, enclosing and protecting internal components, and is constructed from medical-grade materials such as ABS plastic, polycarbonate, or biocompatible polymers.

The first end of the body is configured to connect securely to the air intake port of a CPAP machine through an input fastener, which may include threaded connectors, clamps, rubber coupler, or snap-fit mechanisms. Alternative fastener designs, such as bayonet mounts or quick-connect couplings, provide an airtight and reliable connection. The second end interfaces with CPAP tubing via an output fastener, utilizing compression fittings, threaded connectors, or friction locks to ensure a secure and leak-free attachment. The rubber coupler will allow the device to slide into the machine, similar to the current method in which the hose slides into the machine.

An interior space within the body channels airflow while maintaining the pressure and flow consistency required for CPAP therapy. This interior includes a receiving area designed to hold a replaceable fragrance cartridge securely. The cartridge contains hypoallergenic, medical-grade fragrances, such as essential oils, formulated to promote respiratory relief or relaxation. Fragrance diffusion is facilitated through openings in the cartridge, such as porous membranes or vent systems.

The receiving area is enclosed by a lid that ensures an airtight seal using elastic, hypoallergenic materials. The lid is secured by a locking mechanism, which may include a snap-fit latch, magnetic closure, or threaded ring. In some versions, the lid is transparent or translucent, allowing users to monitor the fragrance cartridge without opening the device.

During use, the device connects to the CPAP machine and tubing, allowing air to flow through the interior space and over the fragrance cartridge. The airflow diffuses scent molecules into the air stream, delivering them to the user and enhancing the therapeutic experience. Depleted cartridges can be easily replaced, ensuring minimal disruption to therapy.

The invention also encompasses a method of use that includes connecting the device to a CPAP machine and tubing, inserting the fragrance cartridge, securing the lid, and operating the CPAP machine to diffuse the fragrance. Cartridge replacement is designed to be simple and efficient, supporting ease of use and continued functionality.

Thus, the continuous positive airway aromatherapy device offers significant benefits, attaching directly to a CPAP housing and featuring an input port, an output port, and a slot for interchangeable fragrance cartridges such as menthol, peppermint, or lavender, which help alleviate congestion and promote relaxation. The snap lid maintains an airtight seal to preserve CPAP machine performance while facilitating easy cartridge replacement. By introducing therapeutic aromas into the airflow, the device enhances user comfort and respiratory therapy effectiveness, particularly during episodes of nasal congestion. This innovation improves user satisfaction and promotes consistent CPAP usage, addressing challenges such as nasal obstruction and supporting better health outcomes. Accordingly, the device addresses limitations present in conventional CPAP machines known in the art.

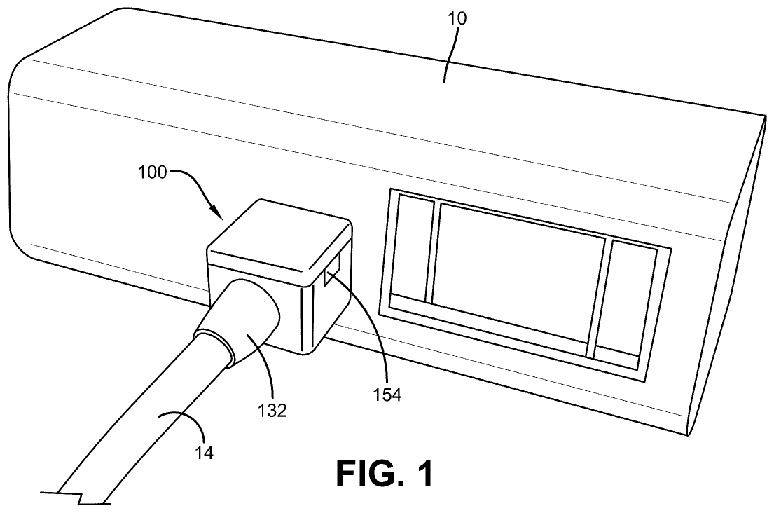

Referring initially to the drawings, FIG. 1 illustrates a perspective view of one potential embodiment of a continuous positive airway aromatherapy device 100 of the present invention attached to a CPAP machine in accordance with the disclosed architecture. The device 100 is designed to integrate with CPAP machines 10, enhancing a user's sleep therapy experience through therapeutic aromatherapy. The body 110 of the device 100 serves as the primary housing, comprising and protecting all internal components. The body 110 is preferably manufactured from medical-grade materials such as but not limited to ABS plastic, polycarbonate, or other biocompatible polymers.

The first end 120 of the body 110 is configured to connect securely to the air intake port 12 of the CPAP machine 10, as seen in FIG. 1. This connection is achieved using at least one input fastener 122, which may include a threaded connector, clamp, or snap-fit fastener. Alternative embodiments of the fastener 122 may include bayonet mounts or quick-connect couplings to enhance ease of use. The input fastener 122 ensures an airtight and reliable connection, preventing air leakage and preserving the integrity of the CPAP machine's 10 airflow.

A second end 130 of the body 110 interfaces with the CPAP tubing 14, as seen in FIG. 1. This connection is facilitated by at least one output fastener 132, designed to provide a secure and leak-free attachment. Output fasteners 132 may include but are not limited to compression fittings, threaded connectors, or friction locks, with variations such as push-to-connect mechanisms or integrated gasket seals to enhance user convenience.

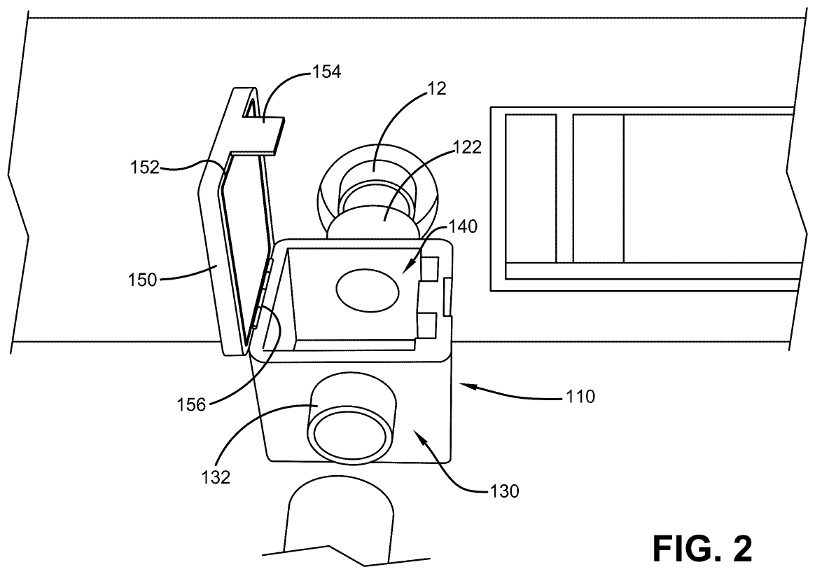

The body 110 contains an interior space 140, as seen in FIG. 2, which directs airflow through the device 100 while maintaining the consistent pressure and flow required for effective CPAP therapy. Within the interior space 140 is at least one receiving area 142, such as a slot or cavity, designed to hold at least one fragrance cartridge 160 securely.

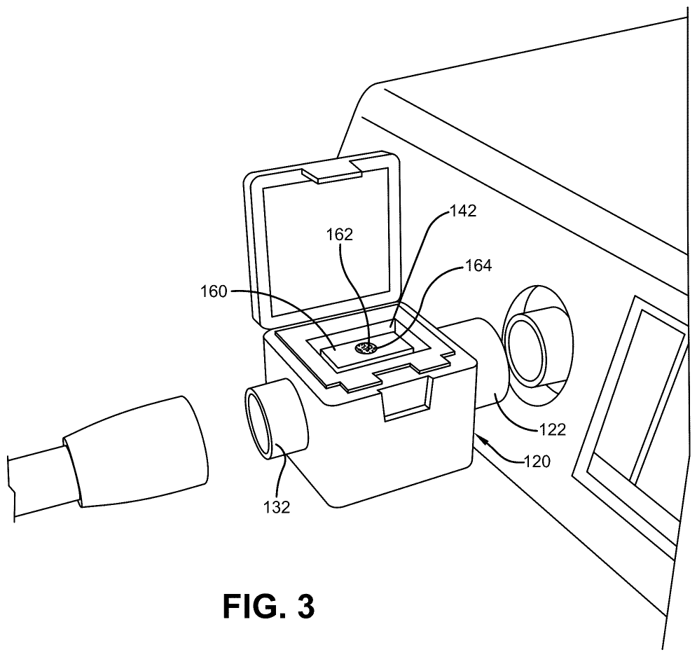

The fragrance cartridge 160 (as seen in FIG. 3) is a replaceable component containing at least one fragrance 162, which may include hypoallergenic, medical-grade essential oils or other inhalable fragrance mediums. These fragrances 162 are available in various formulations, such as but not limited menthol for respiratory relief, lavender for relaxation, or other therapeutic blends to suit individual preferences. The cartridge 160 may feature at least one opening 164, which may take the form of a porous membrane, perforated film, or vent system, enabling controlled diffusion of the fragrance 162 into the airflow. In other embodiments, the cartridge itself emits the fragrance 162, wherein the cartridge 160 may be degradable.

To protect the fragrance cartridge 160 and maintain the device's 100 airtight integrity, the receiving area 142 is covered by a lid 150. The lid 150 is connected to the body 110 by a hinge 156 in one embodiment, allowing it to open and close easily, while alternative configurations may use a snap-fit lid. To ensure an airtight seal, the lid 150 is equipped with at least one seal 152 made of an elastic, hypoallergenic material such as food-grade silicone or thermoplastic elastomers.

The lid 150 is secured during operation by a lid locking member 154 as seen in FIG. 1, which may be a snap-fit latch, magnetic closure, or threaded locking ring. In some embodiments, the lid 150 is constructed from a transparent or translucent material, enabling users to monitor the fragrance cartridge 160 status without opening the device 100.

In operation, the first end 120 is connected to the air intake port 12 of the CPAP machine 10, and the second end 130 is attached to the CPAP tubing 14. Once the fragrance cartridge 160 is inserted into the receiving area 142, the lid 150 is closed and secured. Air from the CPAP machine 10 flows through the interior space 140, passing over the fragrance cartridge 160. The airflow carries scent molecules released from the fragrance 162 through the tubing 14 and delivers them to the user, enhancing the therapeutic experience.

When the fragrance 162 is depleted, the lid 150 can be opened via the hinge 156 or an alternative mechanism to replace the fragrance cartridge 160. The replacement process is designed to be quick and intuitive, minimizing disruption to the user's therapy routine.

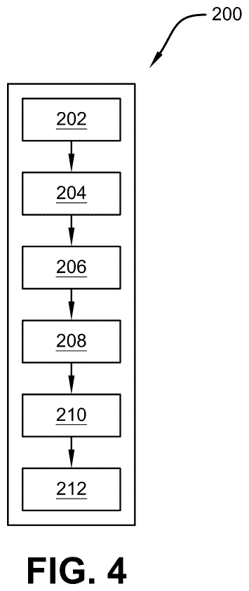

The present invention is also comprised of a method of using 200 the device 100, as seen in FIG. 4. First, a device 100 is provided, comprised of a body 110 with a first end 120, a second end 130, an interior space 140, a receiving area 142, a lid 150, and a fragrance cartridge 160 [Step 202]. Then, the first end 120 of the device 100 can be connected to the air intake port 12 of a CPAP machine 10 using at least one input fastener 122, and the second end 130 can be connected to CPAP tubing 14 using at least one output fastener 132 [Step 204]. Next, the fragrance cartridge 160 can be inserted into the receiving area 142 within the interior space 140, ensuring it is properly aligned and secured in place [Step 206]. After the fragrance cartridge 160 is inserted, the lid 150 can be closed and secured using the lid locking member 154, creating an airtight seal via at least one seal 152 [Step 208]. Once assembled, the CPAP machine 10 can be operated to initiate airflow through the interior space 140 of the device 100 [Step 210]. As air flows through the device 100, it interacts with the fragrance cartridge 160, diffusing scent molecules from the fragrance 162 into the airflow, which is then delivered to the user via the CPAP tubing 14. When the fragrance 162 within the cartridge 160 is depleted, the lid 150 can be opened using the hinge 156 or an alternative mechanism, the depleted fragrance cartridge 160 can then be removed, a new cartridge 160 can be inserted into the receiving area 142, and the lid 150 can be closed and re-secured to restore the device 100 to an operational state [Step 212].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “continuous positive airway aromatherapy device” and “device” are interchangeable and refer to the continuous positive airway aromatherapy device 100 of the present invention.

Notwithstanding the forgoing, the continuous positive airway aromatherapy device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the continuous positive airway aromatherapy device 100 as shown in the FIGS. are for illustrative purposes only, and that many other sizes and shapes of the continuous positive airway aromatherapy device 100 are well within the scope of the present disclosure. Although the dimensions of the continuous positive airway aromatherapy device 100 are important design parameters for user convenience, the continuous positive airway aromatherapy device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A continuous positive airway aromatherapy device comprising:
a body having a first end configured to connect to an air intake port of a CPAP machine and a second end configured to connect to a CPAP tubing;
an input fastener positioned at the first end for securing an airtight connection to the air intake port of the CPAP machine;
an output fastener positioned at the second end for securing an airtight connection to the CPAP tubing;
an interior space within the body for directing airflow between the first end and the second end;
a receiving area within the interior space for holding a fragrance cartridge, the fragrance cartridge configured to release a fragrance into the airflow passing through the interior space; and
a lid attached to the body, the lid configured to cover the receiving area and maintain an airtight seal.

2. The continuous positive airway aromatherapy device of claim 1, wherein the input fastener is comprised of a threaded connector, clamp, or snap-fit fastener.

3. The continuous positive airway aromatherapy device of claim 1, wherein the output fastener is comprised of a compression fitting, threaded connector, or friction lock.

4. The continuous positive airway aromatherapy device of claim 1, wherein the lid is connected to the body by a hinge.

5. The continuous positive airway aromatherapy device of claim 1, wherein the receiving area is comprised of a slot or a cavity.

6. The continuous positive airway aromatherapy device of claim 1, wherein the lid is comprised of a transparent or a translucent material to allow visual inspection of the fragrance cartridge when placed within the interior space and when the lid is closed.

7. The continuous positive airway aromatherapy device of claim 1, wherein the fragrance cartridge comprises a porous membrane, a perforated film, or a vent system to enable a controlled diffusion of the fragrance.

8. The continuous positive airway aromatherapy device of claim 1, wherein the lid is comprised of a lid locking member.

9. A continuous positive airway aromatherapy device comprising:
a body having a first end configured to connect to an air intake port of a CPAP machine and a second end configured to connect to a CPAP tubing;
an input fastener positioned at the first end for securing an airtight connection to the air intake port of the CPAP machine;
an output fastener positioned at the second end for securing an airtight connection to the CPAP tubing;
an interior space within the body for directing airflow between the first end and the second end;
a receiving area within the interior space for holding a fragrance cartridge, the fragrance cartridge configured to release a hypoallergenic essential oil fragrance into the airflow passing through the interior space; and
a lid attached to the body, the lid configured to cover the receiving area and maintain an airtight seal.

10. The continuous positive airway aromatherapy device of claim 9, wherein the hypoallergenic essential oil fragrance is comprised of a menthol or a lavender.

11. The continuous positive airway aromatherapy device of claim 9, wherein the airtight seal is comprised of an elastic hypoallergenic material.

12. The continuous positive airway aromatherapy device of claim 11, wherein the elastic hypoallergenic material is comprised of a food-grade silicone.

13. The continuous positive airway aromatherapy device of claim 11, wherein the elastic hypoallergenic material is comprised of a thermoplastic elastomer.

14. The continuous positive airway aromatherapy device of claim 9, wherein the lid locking member is comprised of a snap-fit latch, a magnetic closure, or a threaded locking ring.

15. The continuous positive airway aromatherapy device of claim 9, wherein the fragrance cartridge comprises a porous membrane, a perforated film, or a vent system to enable a controlled diffusion of the hypoallergenic essential oil fragrance.

16. The continuous positive airway aromatherapy device of claim 9, wherein the input fastener is comprised of a threaded connector, clamp, or snap-fit fastener.

17. The continuous positive airway aromatherapy device of claim 9, wherein the output fastener is comprised of a compression fitting, threaded connector, or friction lock.

18. The continuous positive airway aromatherapy device of claim 9, wherein the lid is connected to the body by a hinge.

19. A method of using a continuous positive airway aromatherapy device, the method comprising the following steps:
providing a continuous positive airway aromatherapy device comprised of a body with a first end, a second end, an interior space, a receiving area, a lid, and a fragrance cartridge;
connecting the first end of the body to an air intake port of a CPAP machine using an input fastener;
connecting the second end of the body to CPAP tubing using an output fastener;
inserting the fragrance cartridge into the receiving area;
closing and securing the lid to create an airtight seal; and
operating the CPAP machine to initiate an airflow through the device, wherein the airflow interacts with the fragrance cartridge to release scent molecules into the airflow and deliver them to the user.

20. The method of using a continuous positive airway aromatherapy device of claim 19 further comprised of a step of replacing the fragrance cartridge when the fragrance is depleted by opening the lid, removing the depleted cartridge, inserting a new fragrance cartridge, and resecuring the lid.
